(19) United States
(12) Patent Application Publication
(10) Pub. No.: US 2023/0255548 A1
(43) Pub. Date: Aug. 17, 2023

(54) METHOD FOR DIAGNOSING AND RESTORING REFLEX MUSCLE ACTIVITY

(71) Applicant: VELES HOLDING AG, Moscow (RU)

(72) Inventors: Jose LEVER PALOMAR, Guadalajara, Jalisco (MX); Maria Sergeevna SVET, Moscow (RU)

(21) Appl. No.: 17/629,451

(22) PCT Filed: Jan. 30, 2020
(86) PCT No.: PCT/RU2020/050011

§ 371 Date: Jan. 24, 2022

(30) Foreign Application Priority Data
Jul. 29, 2019 (RU) 2019123942

(51) Int. Cl. A61B 5/00

(52) U.S. Cl. CPC A61B 5/4519

(57) ABSTRACT

The invention relates to reflex therapy. A method includes searching for and determining priority zones of altered receptor activity and associated muscles with impaired reflex activity, making a preliminary diagnosis of a primary zone, selecting indicator muscles and tightening a tendon of the muscle which participates in a step pattern, searching for a primary zone and determining a stimulus of a damaging modality for the primary zone, verifying the correctness of the determination of the primary zone and stimulus therefor, searching for a compensatory zone and determining a stimulus therefor, stimulating the primary zone and the compensatory receptor zone by simultaneously applying stimuli with the provocation of a deep tendon reflex. Technical result: providing accuracy in discovering zones with impaired reflex activity, determining stimuli for any type of mechanoreceptor and nociceptor damage and providing full restoration of muscle reflex activity.

### FIELD OF INVENTION

The invention relates to medicine, namely to reflex therapy, and can be used to treat patients with somatic-somatic and somatic-visceral disorders of muscular reflex activity in the event of functional problems of the musculoskeletal system with chronic pains, recovery of optimal statics and optimal dynamics, prevention of (sports-related and other) injuries, recovery after injuries and surgeries, and to improve the physical performance of individuals involved in sport activities.

### BACKGROUND

Work with patients suffering from chronic pain syndromes due to malfunctioning of musculoskeletal system often causes great difficulties. In spite of a large number of existing drug and non-drug therapies, a positive effect in treatment cannot be always achieved. The etiology of chronic pains without damage to the corresponding structures is still unknown.

One of the causes of chronic pains of the musculoskeletal system without any obvious lesion focus, such as a hernia that entraps nerves, without any obvious degenerative and inflammatory processes, may be altered reflex muscular activity (a protective muscular reflex response) to previous injuries, surgeries, burns, etc. The protective muscular reflex response occurs in the event of increased sensitivity of the receptor fields in the injured area (scars, burns, fractures, contusions, sprained ligaments). A supersensitive receptor zone (with an increased threshold of excitation) can result in an inadequate reflex response (hypotension of associated muscles and muscle hypertension—as a result of the overload of compensatory muscles). Due to functional weakness, the associated muscles in the injured zone will have a hypo- or hyper-myotatic reflex, which will result in change in statics and dynamics and in overloading other regions, which will compensate for the weakness of the injured weakened region. This will result in an impairment of the musculoligamentary (somato-somatic), somato-visceral reflexes and disrupt the overall sensory-motor balance, which may ultimately result in decompensation and pain symptoms. If the signal from the altered receptor zone causes functional weakness of the associated muscles, then in order to correct, the protective reflex should be changed to the neutral one, which will recover the sensory-motor balance of the region, therefore, the need for compensatory mechanisms will disappear and the optimal statics and dynamics will be recovered.

In order for the protective muscular reflex to be changed to the neutral one, it is necessary to determine a priority receptor zone that causes the protective adaptive reflex response, the modality of the stimulus that has directly caused the protective reflex response of the associated muscles, to determine the compensatory receptor zone and the modality of its damaging stimulus. Where the biomechanical load is redistributed to the regions of the body due to altered reflex activity, receptor zones are formed in the injured zones, the muscles associated with such receptor zones will have a hypo- and hyper-myotatic reflex alterations, which is necessary to maintain altered (non-optimal) statics and balance.

If injuries occur in various regions of the body, a natural protective reaction of the body occurs and the load from the injured body region is distributed to other healthy regions to ensure the recovery of the damaged region. As the injury heals, the muscular reflex activity of the damaged region should be recovered. If for some reasons this does not occur and the sensory signal from the injury zone does not change, then the region will remain in the protective reflex mode causing the need for compensation—redistribution of the load, which results over a long period of time in overloading of the compensatory regions, instability of the regions, pain symptoms and spasms, limited mobility and, as a result, in degenerative changes in tissue structures.

A method for measuring muscle spasticity is known, that comprises immobilizing a muscle, placing a tapping component over a tendon tied to the muscle, in order to establish a tapping point, moving the tapping element to an indentation position at a predetermined distance from said point. After that, the effort of preload on the tapping component is measured in the indentation position and the area is tapped one or more times, the dependence of the reflex response time on the effort applied on the tapping component is determined, the presence or absence of a spastic response is recorded for each tap or series of taps based on predefined criteria related to the times of reflex responses. If no spastic response is detected, then the tapping component is moved to a new position and the above steps are repeated. If a spastic response is detected, then at least one of the tapping points and the effort of the preload are used to determine the threshold of muscular spasticity (WO2012030987 A2, Aug. 8, 2012).

The described diagnostics can be used to determine the dosage of drugs for patients; however, the existing drug therapies cannot provide a quick and irreversible elimination of the increased activity of the tendon reflex and muscle hypertonia that typically occur in the event of muscular spasticity.

The closest analogue is a method for controlling (diagnosing and recovering) the reflex response of muscles of a lumbar spine, that comprises strengthening the muscles with a weak reflex response and inhibiting the reflex response of muscles that compensate for weak muscles. The reflex response of muscles is facilitated as follows: using electromyography, muscles with a decreased reflex response are determined; a zone (with impaired receptor activity) is found which being pressed the reflex activity of muscles with a reduced reflex response is temporarily recovered, and a pressure-applying member is applied to the said zone in order to apply pressure so that the pressure-applying member provides external pressure of not more than 200 mm Hg on the skin mechanoreceptors. The reflex response of muscles is inhibited as follows: muscles that need to be relaxed, for example, due to spasm are found, and to inhibit the reflex response of these muscles, a pressure-applying member is applied to this zone so that the member provides external pressure of at least 400 mmHg on the joint mechanoreceptors (U.S. Pat. No. 5,769,803 A, 6.23.1998).

The disadvantages of the prototype method can include the following. This method provides only a temporary recovery of the reflex activity of the muscles by long wearing of a device containing pressure-applying members on the living body; it provides the recovery of reflex activity of the muscles if the reflex response of the mechanoreceptors only is damaged (not that of nociceptors). If there is a primary zone of altered reflex activity, a secondary (compensatory) zone also occurs, which is formed in the event of an altered signal from the primary zone in order to maintain optimal body biomechanics. However, the prototype method does not include the step of searching for this zone. Moreover, it is impossible to completely recover sensory-motor balance without an exact determination of the damaging stimulus, which caused a change in the reflex activity of the muscles and formed a particular muscle pattern—a protective response to the stimulation of particular muscle fibers.

### SUMMARY

The technical result of the invention proposed is:

the accurate detection of receptor zones with impaired reflex activity and the related associated muscles,

the determination of stimuli of a damaging modality for the primary and compensatory zones for any types of functional damage to mechanoreceptors and nociceptors,

the full recovery of muscular reflex activity and range of motion in the event of injuries of both mechanoreceptors and nociceptors,

an expanded scope of application for the method for recovering muscular reflex activity,

a full and irreversible recovery of the range of motion and stability of the joints.

The method further allows reducing or completely eliminating pain symptoms.

The technical result is achieved by the proposed method for the diagnosis and recovery of reflex muscular activity comprising:

examination and interview of the patient, the detection of damaged receptor zones and the associated muscles with impaired reflex activity by testing,

preliminary diagnosis of the primary receptor zone with impaired reflex activity in the area of the said muscles by applying to it a single stimulus of a damaging modality, during which a temporary recovery of the reflex activity of associated muscles with impaired reflex activity is observed,

the selection of an indicator muscle and clamping the tendon of the muscle involved in the pattern of the step,

searching for the primary zone with impaired reflex activity and determining a stimulus of a damaging modality for it,

check whether the primary zone and the stimulus of the damaging modality for it are correctly determined,

searching for the compensatory receptor zone with impaired reflex activity and determining the stimulus of a damaging modality for it,

stimulation of the primary and compensatory zones with respective damaging stimuli and the recovery of reflex muscular activity,

wherein the primary and compensatory zones are searched for by exposing the region of the receptor zones and associated muscles to single and/or double stimuli of a damaging modality; the primary zone is searched for by applying a single stimulus of a damaging modality to the previously diagnosed primary zone; the hyporeflexia of the indicator muscle is used to determine whether the primary zone is determined correctly; after that, the tendon of the muscle involved in the pattern of the step is released; and when a double damaging stimulus is applied to the primary zone, the hyporeflexia of the indicator muscle is used to determine whether the stimulus of a damaging modality is selected correctly; a compensatory zone is searched for with a tendon clamped by applying a single stimulus of a damaging modality to the region of receptor zones and associated muscles with impaired reflex activity during the application of a double stimulus of a damaging modality to the primary zone, the disappearance of the hyporeflexia of the indicator muscle is used to determine whether the compensatory zone is determined correctly; the temporary recovery of the normal myotatic reflex of the indicator muscle is used to determine whether the stimulus of a damaging modality is selected correctly; the primary and compensatory zones are stimulated by the simultaneous application of the stimuli of a damaging modality determined for them, during which a deep tendon reflex is provoked.

When examining a patient, the area of the muscles is first examined where there have been injuries. During the examination and interview of the patient, complaints of pain are established, a history of all injuries whenever sustained is collected, injured receptor zones with impaired activity are detected, pain symptoms are assessed on a scale of 1 to 10, the mobility and stability of joint are assessed, the symmetry of body regions is assessed, pain symptoms are assessed with a change in body position (when lying down, sitting, standing, in a passive and active movement). Examining a patient can also involve analyzing the data of X-ray studies for structural pathologies. Examining a patient can involve differential diagnostics.

Pain syndromes are assessed on any standard pain scale. For example, a visual analogue pain scale can be selected.

The visual analogue pain scale (VAS) is a 10 cm long straight-line segment. Its beginning corresponds to the lack of pain sensation—“no pain” and the end point means excruciating insupportable pain—“unbearable pain”. The line can be both horizontal and vertical. The patient is encouraged to make on it a mark corresponding to the intensity of the pain they are currently experiencing. The distance from the beginning of the segment (“no pain”) to the mark made is measured in centimeters and rounded to the nearest whole. Each centimeter on the line corresponds to 1 score. At a mark of up to 2 cm, the pain is classified as mild, from 2 to 4 cm—as moderate, from 4 to 6 cm—as severe, from 6 to 8 cm—as the strongest and up to 10 cm—as unbearable. In general, all patients, including children over 5 years old, easily learn to use the VAS.

To determine the associated muscles with impaired reflex activity, in the beginning, the patient is examined and interviewed and the specified muscles are diagnosed using testing. The testing is performed by the muscle test (muscle testing) and/or electromyography.

Muscle testing can be performed, for example, by showing parallel strips and a cross to the patient. It is empirically found that when the patient is looking at parallel lines, all muscles give a hypotonic response, and when a patient is looking at a cross, all muscles should show a strong response, which is associated with fixing the gaze: if the patient is looking at parallel lines, there is a movement of vertical saccades, which informs the nervous system about a supposed movement, information from the visual system contradicts information from the proprioceptive system (there is no signal of motion from muscles or ligaments) and general functional muscle weakness occurs in response to the contradiction. When the patient is looking at the cross, there is a fixation point (cross) and there is no conflict of pieces of information. This method can be used to check the normotonicity of a muscle.

Testing allows identifying associated muscles with impaired reflex activity and supposedly establishing the areas of receptor zones with altered sensitivity.

General muscle hypertension—general hypertonia indicates that all muscles are hyperreflexive. This is a general protective response to stress of any etiology—emotional, injury, toxicity, drug administration, etc.

General muscle hypotension means that all muscles are tested as weak.

To preliminarily diagnose the primary zone with impaired receptor activity, a single stimulus of a damaging modality is applied to it in the form of stroking, which has an effect on mechanoreceptors, or in the form of applying pressure, which has an effect on nociceptors.

When applying a single stimulus of a damaging modality in order to preliminarily diagnose, the temporary recovery of the reflex activity of associated muscles with impaired reflex activity is determined based on the fact that the muscle response changes to normotonic.

Any muscle that is not associated with muscles with impaired reflex activity can be selected as an indicator muscle, wherein such muscle can be selected from the group consisting of: the rectus muscle of thigh, biceps muscle of the arm, the deltoid muscle.

The primary and compensatory zones are searched for with a clamped tendon of the muscle involved in the pattern of the step, which is selected from the group consisting of: the tendon of the sternocleidomastoid muscle, the tendon of the rectus muscle of thigh, the biceps tendon, the Achilles tendon.

The primary and compensatory zones can be searched for with two reciprocal tendons clamped. Clamping the two reciprocal tendons of the muscles involved in the pattern of the step also serves to search for the primary and secondary zones as an additional method of checking for the correct determination of the zones. Clamping of the two tendons and a single stimulus to the zone with altered receptor activity will result in the weakness of all indicator muscles, and a single stimulus for the compensatory zone will recover the weakness of the indicator muscle.

Two simultaneously clamped tendons can be used to identify an area with altered reflex activity (both primary and secondary), and for the primary diagnosis of an impaired movement pattern.

After that, the primary zone (the zone of primary dysfunction)—the zone of increased receptor activity, a signal from which creates a change in the reflex of associated muscles is searched for.

If, when the area under study is being stimulated by stroking (rough touch stimulus) or applying pressure (Pacinian receptor stimulus), the reflex response of the associated muscles is recovered, then this zone is a zone associated with altered reflex activity, and this zone can be either a primary or compensatory (secondary) zone of altered receptor activity (a zone of secondary dysfunction), i.e., a zone that is formed in the event of an altered signal from the primary zone, in order to maintain optimal body biomechanics, to reduce and distribute the level of the excess signal over the segments of the spinal cord.

The mechanoreceptors (fibers of type A (beta), fibers B) are known to excite muscle fibers C (fibers of pain sensitivity) in the event of a rough touch (stroking). In mechanoreceptors, the conduction path is the dorsal columns, fast myelinated fibers in the posterior spinal cord. Signals from the mechanoreceptors of muscles, ligaments and joints are transferred via dorsal columns (S.A. Vorotnikov. Information devices of robotic systems.—M.: PH of from the Bauman MSTU, 2005.—384 p., pages 22, 28).

Nociceptors are fibers of type A and C, which conduct pain and temperature sensitivity. Nociceptors have their own conduction paths for signal transmission—the anterolateral system. A decrease in the signal via the conduction path can be provided by the application of pressure (H. R. Schiffman, Sensation and perception/Transl. from English. Z. Zamchuk.—5th ed—St. Petersburg: Piter, 2003.—928 p., page 811).

Thus, if the reflex response of the associated muscles is recovered upon stimulation of this area by stroking, the impaired receptors are mechanoreceptors. If the reflex response of the associated muscles is recovered upon stimulation of this area by application of pressure (compression), the impaired receptors are nociceptors.

Receptor zones of altered activity can be scars of any origin however long ago sustained, tattoos, piercings, places of limb injuries, head areas (effects of injuries and contusions), areas of any fractures on the body, burns, frostbite, laser correction, removed skin warts (mole-marks, blotches), the dento-facial system after any dental interventions, any areas after cosmetic surgery, including injections. In addition, zones of altered activity can be ligaments, tendons, cranial sutures, joints (joint receptors), skin damaged by dry tetters or other skin diseases, the mucous membranes of the mouth, nose and genitals. Receptor zones with altered activity can be areas after any physiotherapeutic treatment—vibration, heat, cold, areas in contact with chemical stimuli, insect stings, snake bites, areas in contact with cold air, the tongue, the ocular mucous membrane.

After detection of the primary zone, a damaging stimulus is determined that caused a violation of reflex activity (stimulus of a damaging modality) and upon stimulation of the receptor zone by which the reflex activity is changed. Such stimuli can be: pressure, vibration, rough touch, delicate touch, pricking, tickling, scratching, surface and deep pressure, stretching, cold, heat, abrasion, blowing air, direction of light to the eyes, odor, taste, sound, a particular limb movement. Receptor zones can be active in static or be observed in dynamics (in the event of a particular movement that causes weakness).

To this end, the primary zone is exposed to a double damaging stimulus. When the correct stimulus is found using electromyography, an increase is observed in the activity of associated muscles with impaired reflex activity. To increase the sensitivity of receptor fields, the tendon of the muscle involved in the pattern of the step: the sternocleidomastoid, rectus muscle of thigh, biceps tendon or Achilles tendon (universal tendon localization) is clamped. After that, the tendon of the muscle involved in the pattern of the step is released, and when a double damaging stimulus is applied to the primary zone, the hyporeflexia of the indicator muscle is used to determine whether the stimulus of a damaging modality is selected correctly, wherein the application of a single stimulus of the damaging modality to the compensatory (secondary) zone temporarily recovers the normal myotatic reflex of the indicator muscle, and the application of a double stimulus to the primary zone leads to hyporeflexia of the indicator muscle.

In one of the embodiments of the proposed method, paired stimuli are used: a stimulus of a damaging modality applied to the primary receptor zone, temporarily recovering the reflex activity of the associated muscles, and an opposite stimulus of a damaging modality applied to the compensatory receptor zone, causing an increase in the reflex activity of the associated muscles.

The above actions make it possible to determine the primary zone with high accuracy and correctly diagnose a damaging stimulus corresponding to this zone. Therefore, this increases the accuracy of determining the primary zone, which, along with the selection of a necessary stimulus of a damaging modality, increases the effectiveness of further treatment.

The double stimulus should belong to the modality identified earlier. If the damaging stimulus that caused the violation of reflex activity is vibration, then the frequency to which damaged receptors respond (for example, Krause receptors respond to a frequency of 512 Hz) should be selected, and a double stimulus can be made with two tuning forks of the same frequency. To this end, the first tuning fork is placed, the second one is placed immediately near, and in this way, double stimulation of the primary zone is performed. If the necessary stimulus is pricking, then two pricking tools can be used or the same tool can be used to weakly prick one time, and then, prick more deeply without removing the tool. To stimulate the stretch receptors (ligaments), stretching is performed and, without stopping the action of the first stimulus, a second stretching is performed.

Then, the compensatory zone is searched for. To this end, the following method has been developed. The supposed compensatory zone is exposed to a damaging stimulus while a double damaging stimulus is applied to the primary zone. It has been experimentally established that while the compensatory zone is being stimulated with a single stimulus, the recovery of the reflex response of a muscle that does not have an associative connection with a muscle with a reduced reflex response (indicator muscle) is observed, then the compensatory zone is found.

If the compensatory zone is similar to the primary zone (for example, both are ligaments), the damaging stimulus for them may be the same or similar. For example, pressure and stretching are similar stimuli, however, different receptor organs respond to them: Golgi ones respond to stretching, and Pacinian ones respond to pressure. A method for distinguishing them is known. When pressure is applied, this always causes stretching, however, if only Golgi receptors are damaged, they will respond if stretching only is applied to them. Pacinian receptors are rapidly adaptive receptors, therefore, their response to a stimulus will only last while the stimulus itself is active. If, while a tendon is clamped, a pressure stimulus is applied and quickly released (rebound effect) and the indicator muscle becomes strong, this indicates that the Pacinian receptor is damaged. If, while a tendon is clamped, a pressure/stretching stimulus is applied and the indicator muscle becomes weak, this indicates an altered signal from the stretch receptors—Golgi ones.

If these zones are dissimilar (for example, the primary zone is a scar, and the secondary zone is a ligament), then different damaging stimuli are selected for them. To this end, while the compensatory zone is being searched for, various stimuli of the damaging modality are applied to it with a double stimulus of the damaging modality being applied to the primary zone; and the recovery of the reflex response of the muscle that does not have an associative connection with a muscle with reduced reflex activity is observed.

The most common pairs of stimuli of the damaging modality for the primary and secondary zones are: pricking—patting, patting—stroking, cold—patting or pricking, heat—patting or pricking, 128 Hz vibration—pressure, 256 Hz vibration—pressure, 512 Hz vibration—pressure, stretching—stretching (ligaments), stretching—pressure, smelling—pricking, patting—smelling, delicate touch (3 mm touch)—rough touch (patting), rough touch—pricking, delicate touch—pricking.

According to the proposed method, the primary and compensatory zones are simultaneously exposed to stimuli of a damaging modality selected for them, and a deep tendon reflex is provoked, if needed, many times.

A deep tendon reflex can be provoked with a small hammer or hand.

After a compensatory zone is detected, the primary and compensatory zones are simultaneously exposed to stimuli of a damaging modality diagnosed for them, and a deep tendon reflex is provoked with a small hammer or hand. This provokes the central nervous system to adjust the central mechanisms of pain, switch the protective reflex muscular response to a functional one at the level of the respective segment of the spinal cord, which aligns receptor activity of the zones involved in the dysfunction. Thus, the reflex activity of the associated muscles is recovered. The simultaneous stimulation of the two zones and the induction of a deep tendon reflex change the protective reflex of the associated muscles to an optimal one; previously hyporeflexive muscles change their activity to physiological one, recovering the stability of the region and eliminating the need for compensation. Where a knee reflex is reduced, the chin jerk and blink reflexes can be used.

Other factors also affect the diagnosis and adjustment (treatment):

The modality is incorrectly found;
The compensatory zone is incorrectly found;
The patient took muscle relaxants before examination;
The patient has acute inflammation and fever;
The patient takes drugs;
Alcohol intoxication.

Structural pathology (fresh injuries, inflammation, mechanical compression of nerve terminations, degenerative structural changes).

When initially weak muscles are tested after correction, they should change the reflex response to a strong one.

If the reflex response of the muscles has not changed, this indicates that the zones of altered activity and/or stimuli of the damaging modality are incorrectly determined. In this case, the diagnosis of disorders and treatment are repeated until the reflex activity of the associated muscles is fully recovered.

In the event of a muscle hyper response during continuous stimulation of muscle fibers with gamma motor neurons in response to a muscle receptor signal, it cannot relax.

In the event of a hypo-response, muscle innervation cannot be in principle activated with alpha motor neurons in response to signals from the muscle receptors. In the event of a hypo-response, the agonistic muscle responsible for a particular movement does not begin functioning; instead, a group of synergists performs the movement, which violates the pattern of optimal dynamics and leads to compensations, which results in a violation of optimal statics and dynamics and subsequently results in overloading of the joints due to suboptimal work of muscle groups and further to pain symptoms and degenerative processes of tissues.

In addition, there are dysfunctions in which the muscles are initially tested as strong, but after 2-3 provocations they become hyporeflexive, but pains are usually absent. With such disorders, patients, for example, cannot push up more than 2-3 times, since they feel tired. Often, such patterns are associated with peripheral nerve entrapment, and in this case, the patient receives therapy for 2-3 weeks once a week until muscle activity is recovered. Normally, the muscle should perform 10-15 contractions without the reflex becoming weaker.

The proposed method provides the diagnosis and recovery of muscles with hypo- or hyper-reflex activity in various parts of the human body by changing the protective myotatic stretch reflex to a neutral one, recovers normoreflexivity, i.e., recovers the optimal functioning of the muscle and muscle groups—agonists, antagonists, synergists, stabilizers, an increase in the strength of weak muscles and a decrease in the strength of compensatory muscles associated with dysfunction, and, as a result, recovers the compensatory reserve of the body.

### DETAILED DESCRIPTION OF THE PREFERRED EMBODIMENTS

### Exemplary Embodiments

### Example 1

A female patient complained of pain in the right lumbar region which she had suffered from for 6 months when performing gymnastic exercises. The previous injuries included a fracture of the olecranon process of the right ulnar bone sustained 3 years ago.

When examining the right lumbar region, there are no visible edema or deformities, palpation is painless, she has a full range of active motions in the lumbar region with no pain. During the gymnastic exercise with arm load, a sharp pain occurs in the right lumbar region, which prevents from motion performance; instability in the right elbow joint is visually determined.

Differential diagnostics was performed. To this end, the patient was asked to repeat this exercise and the right elbow joint was stabilized with their hand, in this situation the element is performed in full and no pain occurs.

The muscles associated with the right elbow joint were tested using muscle testing, and hyporeflexia of the biceps and triceps muscles of the right shoulder was determined. Compression of the fracture area of the right olecranon process for 3-5 seconds recovered the myotatic reflex in these muscles, which may correspond to the primary zone with impaired reflex activity, where nociceptors are damaged.

The rectus muscle of the left thigh was selected as an indicator muscle. After that, the tendon of the left sternocleidomastoid muscle was clamped, the primary zone was exposed to a damaging stimulus in the form of pricking, which caused hyporeflexia of the indicator muscle. Hyporeflexia of the indicator muscle was also observed during synchronous pricks with two pricking tools without compression of the tendon. Therefore, the fracture zone is actually the primary zone of increased receptor activity, and pricking is a correctly selected damaging stimulus.

Subsequent patting of the fracture zone recovered the normal reflex activity of weak muscles for 3-5 seconds. Therefore, this stimulus is a correctly diagnosed stimulus of a damaging modality for the compensatory zone.

The pair of dysfunctional receptors was checked for whether it is correctly found: applying a double damaging stimulus to the primary zone in the form of pricking caused hyporeflexia of the indicator muscle, and concurrent application of a single damaging stimulus in the form of patting recovered the normal myotatic reflex of the indicator muscle.

After, the receptor imbalance in the fracture zone was eliminated: the primary and compensatory zones were simultaneously stimulated with appropriate damaging stimuli: the primary zone (the zone of the olecranon process fracture) was pricked, the compensatory zone (lateral epicondyle of the right shoulder) was patted, and a deep tendon reflex was provoked.

This procedure allowed recovering the normal myotatic reflex in the biceps and triceps muscles of the right shoulder. The performance of the gymnastic exercise was complete and painless. At the follow-up examination 2 weeks later, the patient reports the absence of previous complaints, all muscles previously involved in dysfunction were normotonic.

### Example 2

A male patient complained of shoulder pain. He had no previous injuries, however, the patient feels the weakness of the entire limb (arm), the range of motion is deteriorated. The previous history includes a burn of his hand. The burn left a scar, but the patient does not feel any discomfort in the hand.

A muscle test of the forearm muscles showed the weakness of the round pronator, the long head of the biceps and flexors of the wrist. Electromyography showed a hyperresponse of the brachial muscle and the short biceps head.

To find the primary zone, the burn site was clamped, the tested muscles changed the response to a normotonic one. Therefore, damaged receptors are nociceptors, and the burn is the primary zone of altered receptor activity.

After that, the damaging stimulus was determined for the primary zone. The biceps of the right shoulder was selected as an indicator muscle. The tendon of the sternocleidomastoid muscle was clamped. Since the burn was reported in the history, the primary zone was exposed to stimulation with a hot thing—a spoon heated to a temperature of 45° C., which led to hyporeflexia of the indicator muscle, which also occurred even without compression of the tendon when a double damaging stimulus with hot things—spoons heated to a temperature of 45° C. were applied to the primary zone, wherein at first, one spoon was applied to the said zone and, without removing it, the second spoon was immediately applied.

Therefore, the zone of the burn is actually the primary zone of increased receptor activity, and heating is a correctly selected damaging stimulus.

Then, the compensatory zone was found. When a double damaging stimulus is applied to the primary zone in the form of hot spoons with the tendon of the sternocleidomastoid muscle clamped, during which the indicator muscle showed weakness, a stimulus in the form of pricking was applied from above directly in the burn area. A muscle test showed that the strength of the indicator muscle was recovered. Therefore, the compensatory zone is the tissues located in the burn area, and heating for the burn and pricking for the tissues surrounding the burn are correctly selected damaging stimuli.

Then, the primary and compensatory zones were simultaneously exposed to appropriate damaging stimuli: the primary zone (burn) was heated, the compensatory zone was pricked, and a deep tendon reflex was provoked by tapping.

After this procedure, a muscle test of the forearm muscles showed the recovery of the reflex activity of the round pronator, the long head of the biceps and flexors of the wrist. Electromyography showed normotonicity of the brachial muscle and the short biceps head. The pain in the shoulder almost disappeared.

### Example 3

The patient complained of pain in his right knee joint during football games, especially at heavy-duty running exercises. A history of previous injuries includes a fall 1 year ago while skiing. The X-ray showed Degrees 1-2 right-sided gonarthrosis according to Kellgren-Lawrence. Magnetic resonance imaging (MRI) of the right knee showed Degree 1 gonarthrosis, moderate degenerative changes in the anterior cruciate ligament, and partial fraying. Examination of the right knee joint detects a slight smoothness of the joint contours, no visible edemas or deformations, palpation is painful in the projection of the lateral part of the joint space, the pain becomes more intense during sharp jerky movements. Active movements in the right knee joint have full range, are painless. Negative symptom of patellar fluctuation. The anterior drawer signs and Lachman test are slightly positive, the posterior drawer sign is negative, no lateral instability in the knee joint has been identified.

The muscles associated with the right knee joint were tested using muscle testing, and hyporeflexia of the right rectus muscle of thigh and hyporeflexia of hamstrings on the opposite side were determined. Taking into account the mechanism of the injury to the right lower limb sustained a year ago during skiing and the combination of hyporeflexia of the right rectus muscle of thigh and hamstrings on the opposite side, a dysfunction of the anterior cruciate ligament of the right knee joint was assumed. Prior to the test, a dorsal displacement of the proximal tibia was performed, which decreased the tension of the anterior cruciate ligament. This resulted in the recovery of the myotatic reflex in these muscles, which may indicate the presence of a primary zone in the anterior cruciate ligament of the right knee joint.

The biceps muscle of the right shoulder was chosen as the indicator muscle, the tendon of the left sternocleidomastoid muscle was clamped, and the stretching the anterior cruciate ligament was selected as a damaging stimulus to the right knee, reproducing the anterior drawer sign, which leads to hyporeflexia of the indicator muscle that appears during synchronous double shocks even without compression of the tendon. Therefore, the right knee joint is actually the primary zone of increased receptor activity, and stretching the anterior cruciate ligament is a correctly selected damaging stimulus. After, the compensatory zone was found and the stimulus corresponding to it was determined. When a double damaging stimulus is applied to the primary zone, which caused hyporeflexia of the indicator muscle, an anti-stimulus for mechanoreceptors (surface stroking) of the right knee joint lateral ligament was applied, which recovered the normal myotatic reflex of the indicator muscle for a short while, and stretching the lateral ligament of the right knee joint recovered the normal myotatic reflex completely.

After, the receptor imbalance in the fracture zone was eliminated. To this end, the primary and main compensatory zones were simultaneously stimulated with appropriate damaging stimuli: the primary zone (anterior cruciate ligament of the right knee joint) and the compensatory zone (lateral ligament of the right knee joint) were stretched and a deep tendon reflex was provoked.

After this procedure, the normal myotatic reflex of the right rectus muscle of thigh and hamstrings of the left thigh was recovered.

At the follow-up examination 2 weeks later, the patient reported the absence of pain during football games, the previously weak muscles were normotonic.

### Example 4

The female patient complained of pain in the cervical spine, which became more intense when seated. The patient's work is related to a constant sitting position. She had no previous injuries. X-ray showed Degree 2 osteochondrosis of the cervical spine, Degrees 2-3 spondylosis. Asymmetry and a difference in the tone of the neck extensors are observed during the examination of the cervical spine. Palpation is harshly painful in the projection of the nuchal ligament at the level of C2-C3. The muscles associated with the cervical spine were tested using muscle testing and hyporeflexia of left neck extensors was determined in the sitting position and normoreflexia of these muscles was determined in the standing position. With the above complaints and hyporeflexive muscles in a sitting position, dysfunction of the pelvis or lumbar spine is probable. In the sitting position, the capsule of the right sacroiliac joint was stroked and the recovery of the myotatic reflex was observed in these muscles, which may correspond to the primary zone with the damaged Golgi receptor organ in the right sacroiliac joint.

The posterior portion of the left deltoid muscle was selected as an indicator muscle, the tendon of the right biceps was clamped, and a damaging stimulus was applied to the right sacroiliac joint in the form of stretching the joint capsule, which caused the hyporeflexia of the indicator muscle, which was also observed during synchronous double stretching of the capsule without compression of the tendon. Therefore, the right sacroiliac joint is actually the primary zone of increased receptor activity, and stretching the capsule of the right sacroiliac joint is a correctly selected damaging stimulus.

After, the compensatory zone was found and the stimulus corresponding to it was selected. When a double damaging stimulus is applied to the primary zone, which caused hyporeflexia of the indicator muscle, an anti-stimulus for mechanoreceptors (surface stroking) of the nuchal ligament area was applied, which recovered the normal myotatic reflex of the indicator muscle for a short while, and stretching the nuchal ligament recovered the normal myotatic reflex in full.

After, the receptor imbalance in the fracture zone was eliminated. To this end, the primary and main compensatory zones were simultaneously stimulated with appropriate damaging stimuli: the primary zone (sacroiliac joint to the right) and the compensatory zone (nuchal ligament to the left) were stretched and a deep tendon reflex was provoked.

This procedure resulted in the recovery of the normal myotatic reflex of neck extensors Immediately after the treatment, pain was observed to be relieved by 80%. At the follow-up examination 2 weeks later, the patient reported the absence of pain in the cervical spine.

### Example 5

A male patient complained of pain in the right knee joint when walking. He had the following past injuries: he felt discomfort in the right lower limb for 2 weeks 3 years ago when jumping from a height of 3 meters. The X-ray showed Degree 2 right-sided gonarthrosis according to Kellgren-Lawrence. Ultrasonic scanning of the right knee joint showed signs of hypertrophy of the capsule of the superior recess, moderate degenerative changes in the posterior horns of both menisci. Examination of the right knee joint detects a slight smoothness of the joint contours, palpation is painful in the projection of the anteromedial part of the joint space, the pain becomes more intense in a standing position. Active movements in the right knee joint have full range, are painless. Negative symptom of patellar fluctuation. No joint instability is detected.

The muscles associated with the right knee joint were tested using muscle testing, and hyporeflexia of the quadriceps of the right thigh, adductors of the right thigh. Taking into account the axial mechanism of the injury to the right lower limb 3 years ago, tapping was performed through the calcaneal region over the entire right lower limb, which resulted in a deep tendon reflex. This resulted in the recovery of the myotatic reflex in these muscles, which may correspond to the area with primarily disturbed intraarticular paleospinoreticular nociceptor. When the primary zone was stimulated by tapping, the severity of dysfunction decreased.

After, differential diagnostics was performed. To this end, short impacts were made along the axis of the right hip joint and the right ankle joint, which did not provide back hyporeflexia of previously weak muscles, and an impact along the axis of the right knee joint caused hyporeflexia of the right quadriceps and the adductors of the right thigh.

The left rectus muscle of thigh was selected as an indicator muscle, the tendon of the left sternocleidomastoid muscle was clamped, and a damaging stimulus was applied to the right knee joint in the form of impacts along the axis of the right knee joint, which caused the hyporeflexia of the indicator muscle, which was also observed during synchronous double impacts without compression of the tendon. Therefore, the right knee joint is actually the primary zone of increased receptor activity, and short impacts along its axis are a correctly selected damaging stimulus.

After, the compensatory zone was found and the stimulus corresponding to it was determined. To this end, when a double damaging stimulus was applied to the primary zone in the form of a short impact along the axis, which resulted in hyporeflexia of the indicator muscle, a single stimulus was applied in the form of pricking in the area of the anteromedial part of the joint space of the right knee joint, which recovered the normal myotatic reflex of the indicator muscle.

After, the receptor imbalance in the fracture zone was eliminated. To this end, the primary and main compensatory zones were simultaneously stimulated with appropriate damaging stimuli: the primary zone (right knee joint) was tapped along the axis of the joint, and the compensatory zone (anteromedial part of the right knee joint) was pricked and a deep tendon reflex was provoked.

After this procedure, the normal myotatic reflex in the quadriceps and the adductors of the right thigh was recovered. At the follow-up examination 1 week later, the patient reported the absence of previous complaints, the previously weak muscles were normotonic.

### Example 6

A female patient complained of pain in the Achilles tendons for 3 months, without positive changes in the treatment. Her previous injuries included the following: 3 years ago she dropped a heavy thing on her right foot, which resulted in a non-displaced fracture of the 3rd metatarsal bone. X-ray of her right foot showed a consolidated non-displaced fracture of the base of the right 3rd metatarsal bone. Ultrasonic scanning (ultrasound) of the her Achilles tendons showed signs of bilateral achillobursitis. Examination of the region of her Achilles tendons identified the smoothness of the contours and edema, palpation is sharply painful in the projection of the Achilles bursas and the Achilles tendon, the integrity of the tendon is not broken. The patient walks with a hobble. Testing the muscles associated with the ankle joints is not objective since it causes severe pain.

The right biceps was selected as an indicator muscle, the tendon of the left sternocleidomastoid muscle was clamped, and the modality of the broken receptors in the area of the Achilles tendon was determined. To this end, this zone was exposed to various damaging stimuli—pricking, patting and stroking, however, only stroking the right and left Achilles tendons resulted in hyporeflexia of the indicator muscle, which did not appear during synchronous stroking with the tendon non compressed. Therefore, the zone of the clinical manifestations is the compensatory zone of increased receptor activity, and stroking is a correctly selected damaging stimulus. Then, the primary zone was preliminarily searched for. The tendon of the left sternomastoid muscle was clamped and the right Achilles tendon was stroked, which resulted in hyporeflexia of the indicator muscle, and the compression of the area of the metatarsal fracture returned the normoreflexia of the indicator muscle. The same thing happened when the left Achilles tendon was stimulated.

For a more accurate determination of the primary zone, the tendon of the left sternocleidomastoid muscle was clamped and the fracture area was pricked as a damaging stimulus, which resulted in hyporeflexia of the indicator muscle, which also occurred during synchronous pricks with two pricking tools with the tendon non clamped. Therefore, the fracture zone is actually the primary zone of increased receptor activity, and pricking is a correctly selected damaging stimulus.

After, the pair of dysfunctional receptors was checked for whether it was correctly found. To this end, while the tendon of the left sternocleidomastoid muscle was clamped, a damaging stimulus was applied to the primary zone in the form of pricking, which caused hyporeflexia of the indicator muscle. After, a single stimulus was applied in the form of stroking to the compensatory zone in the region of the Achilles tendons, which recovered the normal myotatic reflex of the indicator muscle. Therefore, these areas with clinical manifestations are compensatory, and stimulating only the region of the right Achilles tendon eliminates the hyporeflexia of the indicator muscle caused by double stimulation (pricking) of the fracture area.

After, the receptor imbalance in the fracture zone was eliminated: the primary and main compensatory zones were simultaneously stimulated with appropriate damaging stimuli: the primary zone of the metatarsal fracture to the right was stimulated by pricking, the compensatory zone of the right Achilles tendon was stimulated by stroking and a deep tendon reflex was provoked. After this procedure, the pain decreased by 50%. At the follow-up examination 1 week later, the patient reported the absence of previous complaints, there was no visible edema, there was no palpatory soreness. She walked with full physical load.

Therefore, the correct selection of stimuli of damaging modality increases the effectiveness and accuracy of the diagnosis of disorders in the reflex activity of muscles, since the exact location of the primary and compensatory zones is confirmed during the determination of these stimuli. Stimuli of a damaging modality for the primary and compensatory zones can be determined for any types of damage to mechanoreceptors and nociceptors in various parts of the human body.

Since the proposed method provides for the recovery of muscular reflex activity for any type of damage to mechanoreceptors and nociceptors, the treatment efficacy is increased. The method has a wider scope of application, since it recovers muscle reflex activity with a wide variety of zones of impaired receptor activity, which can be scars of any origin, tattoos, piercings, places of limb injuries, head areas (effects of injuries and contusions), areas of any fractures on the body, burns, frostbite, laser correction, removed skin warts (mole-marks, blotches), the dento-facial system after any dental interventions, any areas after cosmetic surgery, including injections.

As shown by the above clinical trials, the proposed method provides a complete and irreversible recovery of the range of motion and stability of the joints without the need for subsequent wearing of various devices. After treatment, no physiotherapy or painkillers are needed, the patient can load their muscles with exercises, even if they have been hyporeflexive for a long time.

The proposed method increases the compensatory resources of the body, recovers muscle activity and range of motion, relieves pain symptoms.

What is claimed is:

1. A method for diagnosing and recovering of a reflex muscular activity, comprising:
examining and questioning of the patient,
detecting of damaged receptor zones and related associated muscles with an impaired reflex activity by testing,
preliminary diagnosing of a primary receptor zone with the impaired reflex activity by applying a single stimulus of a damaging modality to the primary receptor zone, during which a temporary recovery of the reflex activity of the associated muscles with the impaired reflex activity is diagnosed,
selecting of an indicator muscle and clamping a tendon of the muscle involved in a step pattern,
searching for the primary receptor zone with the impaired reflex activity and determining a stimulus of a damaging modality for it,
checking whether the primary receptor zone and the stimulus of the damaging modality for it are correctly determined,
searching for a compensatory receptor zone with the impaired reflex activity and determining a stimulus of a damaging modality for it,
stimulating of the primary receptor and the compensatory receptor zones and recovering of the reflex muscle activity,
wherein the primary and the compensatory zones are searched for by exposing a region of the receptor zones and the associated muscles with the impaired reflex activity to a single and/or a double stimuli of a damaging modality; wherein the primary zone is searched for by applying a single stimulus of a damaging modality to a previously diagnosed primary receptor zone; and a hyporeflexia of an indicator muscle is used to determine whether the primary zone is determined correctly; after that, the tendon of the muscle involved in the pattern of the step is released; and
when a double damaging stimulus is applied to the primary receptor zone, the hyporeflexia of the indicator muscle is used to determine whether the stimulus of the damaging modality is selected correctly; a compensatory zone is searched for with a tendon clamped by applying a single stimulus of a damaging modality to the region of the receptor zones and the associated muscles with the impaired reflex activity during the application of the double stimulus of the damaging modality to the primary receptor zone, a disappearance of the hyporeflexia of the indicator muscle is used to determine whether the compensatory receptor zone is determined correctly; the temporary recovery of a normal myotatic reflex of the indicator muscle is used to determine whether the stimulus of the damaging modality is selected correctly is determined for the compensatory receptor zone; the primary and the compensatory zones are stimulated by the simultaneous application of the stimuli of the damaging modality determined for them, during which a deep tendon reflex is provoked.

2. The method according to claim 1, wherein during the examining and questioning of the patient, complaints of pain are established, injured receptor zones with impaired activity are detected, pain symptoms are assessed on a scale of 1 to 10, a mobility and a stability of joint are assessed, a symmetry of body regions is assessed, pain symptoms are assessed with a change in body position (when lying down, sitting, standing, in a passive and an active movement).

3. The method according to claim 2, wherein scars of any origin and statute of limitation, tattoos, piercings, head areas after injuries and contusions, areas of any fractures on the body, burns, frostbite, laser correction, removed skin warts (mole-marks, blotches), the dento-facial system after any dental interventions, any areas after cosmetic surgery, including injections, ligaments, tendons, cranial sutures, joints (joint receptors), skin damaged by dry tetters or other skin diseases, mucous membranes of the mouth, nose and genitals, areas after any physiotherapeutic treatment—vibration, heat, cold, areas in contact with chemical irritants, insect stings, snake bites, areas in contact with cold air, tongue, the ocular mucous membrane are identified as injured receptor areas with impaired activity.

4. The method according to claim 1, wherein examining the patient can also involve analyzing a data of X-ray studies for structural pathologies.

5. The method according to claim 1, wherein examining the patient involves differential diagnosis.

6. The method according to claim 1, wherein the testing for determining associated muscles with the impaired reflex activity is performed by a muscle test and/or an electromyography.

7. The method according to claim 1, wherein during the preliminary diagnosing of the primary receptor zone with the impaired reflex activity, the single stimulus of the damaging modality is applied to it in a form of a stroking or applying a pressure.

8. The method according to claim 1, wherein for searching for the primary receptor and the compensatory receptor zones, the single or the double stimulus of the damaging modality is selected from a group consisting of: a pressure, a vibration, a rough touch, a delicate touch, a pricking, a tickling, a scratching, a surface and a deep pressure, a stretching, an application of cold, an application of heat, an abrasion, a blowing air, direction of light to eyes, an odor, a taste, a sound, a limb movement.

9. The method according to claim 1, wherein the tendon of the muscle involved in the pattern of the step is selected from a group consisting of: a tendon of a sternocleidomastoid muscle, a tendon of a rectus muscle of a thigh, a biceps tendon, an Achilles tendon.

10. The method according to claim 1, wherein the primary receptor and the compensatory receptor zones are searched for with two reciprocal tendons clamped.

11. The method according to claim 1, wherein the primary and the compensatory zones are repeatedly exposed to stimuli of the damaging modality selected for them and a deep tendon reflex is provoked.

12. The method according to claim 1, wherein any muscle that is not associated with the muscles with the impaired reflex activity is selected as the indicator muscle.

13. The method according to claim 12, wherein the indicator muscle is a muscle selected from a group consisting of: a rectus muscle of thigh, a biceps muscle of an arm, a deltoid muscle.

14. The method according to claim 1, wherein the deep tendon reflex is provoked by a small hammer or a hand.

15. The method according to claim 1, wherein the primary receptor and the compensatory receptor zones where mechanoreceptors and/or nociceptors are damaged are searched for and stimulated.
